SEQUENCE LISTING (188 sequences)

SEQ ID NO: 1 - DNA, 21 bp, Artificial Sequence
  Synthetic Primer
agctgagaatgacgcacagaa

SEQ ID NO: 2 - DNA, 22 bp, Artificial Sequence
  Synthetic Primer
atatgggctggattactttggc

SEQ ID NO: 3 - DNA, 21 bp, Artificial Sequence
  Synthetic Primer
catgtacgttgctatccaggc

SEQ ID NO: 4 - DNA, 21 bp, Artificial Sequence
  Synthetic Primer
ctccttaatgtcacgcacgat

SEQ ID NO: 5 - DNA, 19 bp, Artificial Sequence
  Synthetic Primer
gtgcatgtggtgtatcgcc

SEQ ID NO: 6 - DNA, 21 bp, Artificial Sequence
  Synthetic Primer
aggacctcgatgttgtagtca

SEQ ID NO: 7 - DNA, 22 bp, Artificial Sequence
  Synthetic Primer
tcaactatttggctttgtcagg

SEQ ID NO: 8 - DNA, 19 bp, Artificial Sequence
  Synthetic Primer
gtgagaatccgtatgcccc

SEQ ID NO: 9 - DNA, 21 bp, Artificial Sequence
  Synthetic Primer
tccatcttgtcgtcttcggaa

SEQ ID NO: 10 - DNA, 19 bp, Artificial Sequence
  Synthetic Primer
gggctggttgttgtcgtgt

SEQ ID NO: 11 - DNA, 22 bp, Artificial Sequence
  Synthetic Primer
ctctgccaaacccttctttagc

SEQ ID NO: 12 - DNA, 22 bp, Artificial Sequence
  Synthetic Primer
cgtgaatagcactgacatccaa

SEQ ID NO: 13 - DNA, 19 bp, Artificial Sequence
  Synthetic Primer
ccatgcaggtgagcttcgt

SEQ ID NO: 14 - DNA, 21 bp, Artificial Sequence
  Synthetic Primer
gaatctgcgagagacaccatc

SEQ ID NO: 15 - DNA, 20 bp, Artificial Sequence
  Synthetic Primer
accccagggacagcatatca

SEQ ID NO: 16 - DNA, 20 bp, Artificial Sequence
  Synthetic Primer
tgcgatccgactcaccaata

SEQ ID NO: 17 - DNA, 23 bp, Artificial Sequence
  Synthetic Primer
cttgatggagaccctcctgtatg

SEQ ID NO: 18 - DNA, 21 bp, Artificial Sequence
  Synthetic Primer
agggtccaggtcctcttcaga

SEQ ID NO: 19 - DNA, 25 bp, Artificial Sequence
  Synthetic Primer
tgctgtctccatgtttgatgtatct

SEQ ID NO: 20 - DNA, 22 bp, Artificial Sequence
  Synthetic Primer
tctctgctccccacctctaagt

SEQ ID NO: 21 - DNA, 23 bp, Artificial Sequence
  Synthetic Primer
tccagaaactaatggcagatccc

SEQ ID NO: 22 - DNA, 22 bp, Artificial Sequence
  Synthetic Primer
aattccctacgctttgggtttt

SEQ ID NO: 23 - DNA, 23 bp, Artificial Sequence
  Synthetic Primer
tgaactgacaacaatcctgaagg

SEQ ID NO: 24 - DNA, 21 bp, Artificial Sequence
  Synthetic Primer
cttgcacgcttttcctcacac

SEQ ID NO: 25 - DNA, 22 bp, Artificial Sequence
  Synthetic Primer
cagaagtcctacaaggtgtcca

SEQ ID NO: 26 - DNA, 21 bp, Artificial Sequence
  Synthetic Primer
ctctggttgaccgtaactgcg

SEQ ID NO: 27 - DNA, 23 bp, Artificial Sequence
  Synthetic Primer
ctcctacccacgcagatttattc

SEQ ID NO: 28 - DNA, 21 bp, Artificial Sequence
  Synthetic Primer
agagtgagaccacgaagagac

SEQ ID NO: 29 - DNA, 23 bp, Artificial Sequence
  Synthetic Primer
tacagacacagtaacccaaagga

SEQ ID NO: 30 - DNA, 21 bp, Artificial Sequence
  Synthetic Primer
tggattgcttgtgaccatgag

SEQ ID NO: 31 - DNA, 23 bp, Artificial Sequence
  Synthetic Primer
acgtggatgattactacgacacc

SEQ ID NO: 32 - DNA, 21 bp, Artificial Sequence
  Synthetic Primer
tgcttttctcacggcatttct

SEQ ID NO: 33 - DNA, 19 bp, Artificial Sequence
  Synthetic Primer
tggggaccacaacatgctg

SEQ ID NO: 34 - DNA, 21 bp, Artificial Sequence
  Synthetic Primer
tcagggcagatgaatgcactg

SEQ ID NO: 35 - DNA, 21 bp, Artificial Sequence
  Synthetic Primer
cactgtctcactacgtgtcgg

SEQ ID NO: 36 - DNA, 19 bp, Artificial Sequence
  Synthetic Primer
ccagccaatggaggtcagc

SEQ ID NO: 37 - DNA, 19 bp, Artificial Sequence
  Synthetic Primer
gcgggagatcgtttcccag

SEQ ID NO: 38 - DNA, 19 bp, Artificial Sequence
  Synthetic Primer
cggcgctcatgtcactctt

SEQ ID NO: 39 - DNA, 21 bp, Artificial Sequence
  Synthetic Primer
acgctatgagacctcactgaa

SEQ ID NO: 40 - DNA, 20 bp, Artificial Sequence
  Synthetic Primer
tcctgggtcaacccctcaag

SEQ ID NO: 41 - DNA, 22 bp, Artificial Sequence
  Synthetic Primer
aatcgtcaatgccagtgtactt

SEQ ID NO: 42 - DNA, 23 bp, Artificial Sequence
  Synthetic Primer
tctcatcgcagtcaggatcataa

SEQ ID NO: 43 - DNA, 19 bp, Artificial Sequence
  Synthetic Primer
gtggcccggatgtgagaag

SEQ ID NO: 44 - DNA, 20 bp, Artificial Sequence
  Synthetic Primer
ggagcccttgtcggatgatg

SEQ ID NO: 45 - DNA, 22 bp, Artificial Sequence
  Synthetic Primer
acaactttggtatcgtggaagg

SEQ ID NO: 46 - DNA, 19 bp, Artificial Sequence
  Synthetic Primer
gccatcacgccacagtttc

SEQ ID NO: 47 - DNA, 21 bp, Artificial Sequence
  Synthetic Primer
cctcccaaaggatgtacccaa

SEQ ID NO: 48 - DNA, 23 bp, Artificial Sequence
  Synthetic Primer
ctctatctccacatacccgttct

SEQ ID NO: 49 - DNA, 21 bp, Artificial Sequence
  Synthetic Primer
agaccccgctatggctctatt

SEQ ID NO: 50 - DNA, 22 bp, Artificial Sequence
  Synthetic Primer
gcttcctctaagtggcacagat

SEQ ID NO: 51 - DNA, 20 bp, Artificial Sequence
  Synthetic Primer
tcagagcctgccaagatcac

SEQ ID NO: 52 - DNA, 22 bp, Artificial Sequence
  Synthetic Primer
cacaagtgtctttaccacctgt

SEQ ID NO: 53 - DNA, 21 bp, Artificial Sequence
  Synthetic Primer
gccaaatcataaacggcgagg

SEQ ID NO: 54 - DNA, 19 bp, Artificial Sequence
  Synthetic Primer
cgcccgagcagaacaattc

SEQ ID NO: 55 - DNA, 19 bp, Artificial Sequence
  Synthetic Primer
ggctcctggcaaaaggtca

SEQ ID NO: 56 - DNA, 20 bp, Artificial Sequence
  Synthetic Primer
ctgcgtagttgtgctgatgt

SEQ ID NO: 57 - DNA, 22 bp, Artificial Sequence
  Synthetic Primer
ctcctgcaagagtttgatgtcc

SEQ ID NO: 58 - DNA, 21 bp, Artificial Sequence
  Synthetic Primer
tcatgccgatgtcatggtagg

SEQ ID NO: 59 - DNA, 20 bp, Artificial Sequence
  Synthetic Primer
cccacactcaggtgatcgag

SEQ ID NO: 60 - DNA, 21 bp, Artificial Sequence
  Synthetic Primer
gagctgctttcgcttagtctt

SEQ ID NO: 61 - DNA, 20 bp, Artificial Sequence
  Synthetic Primer
aaggccgtgtcagaactcaa

SEQ ID NO: 62 - DNA, 20 bp, Artificial Sequence
  Synthetic Primer
gttttccatctcagcctgga

SEQ ID NO: 63 - DNA, 22 bp, Artificial Sequence
  Synthetic Primer
gcacatttccagcccctttaaa

SEQ ID NO: 64 - DNA, 17 bp, Artificial Sequence
  Synthetic Primer
gggcgaggctcatcgat

SEQ ID NO: 65 - DNA, 23 bp, Artificial Sequence
  Synthetic Primer
tacaatacggtgtggagtatgga

SEQ ID NO: 66 - DNA, 21 bp, Artificial Sequence
  Synthetic Primer
tcattggcttaggtggctttg

SEQ ID NO: 67 - DNA, 22 bp, Artificial Sequence
  Synthetic Primer
ggcaggttgtccttctattcag

SEQ ID NO: 68 - DNA, 19 bp, Artificial Sequence
  Synthetic Primer
cagtgtggggcgtaagagt

SEQ ID NO: 69 - DNA, 22 bp, Artificial Sequence
  Synthetic Primer
gtgtgtggacctccatgttatt

SEQ ID NO: 70 - DNA, 20 bp, Artificial Sequence
  Synthetic Primer
tgccccatgacgtgatacct

SEQ ID NO: 71 - DNA, 21 bp, Artificial Sequence
  Synthetic Primer
tgccgcactcagtgttgttag

SEQ ID NO: 72 - DNA, 21 bp, Artificial Sequence
  Synthetic Primer
gcaattcccgcacaagattct

SEQ ID NO: 73 - DNA, 19 bp, Artificial Sequence
  Synthetic Primer
cggagcaaacctcggagtc

SEQ ID NO: 74 - DNA, 21 bp, Artificial Sequence
  Synthetic Primer
gcggccagaaacaatggatag

SEQ ID NO: 75 - DNA, 20 bp, Artificial Sequence
  Synthetic Primer
gctaccccaccgagtacatc

SEQ ID NO: 76 - DNA, 19 bp, Artificial Sequence
  Synthetic Primer
ggctcacgacactgaagca

SEQ ID NO: 77 - DNA, 19 bp, Artificial Sequence
  Synthetic Primer
agcgaacgcacatcaagac

SEQ ID NO: 78 - DNA, 20 bp, Artificial Sequence
  Synthetic Primer
ctgtaggcgatctgttgggg

SEQ ID NO: 79 - DNA, 21 bp, Artificial Sequence
  Synthetic Primer
tcagacgaactcagtgtacgg

SEQ ID NO: 80 - DNA, 21 bp, Artificial Sequence
  Synthetic Primer
cgtagtggacgaggaacatgg

SEQ ID NO: 81 - DNA, 21 bp, Artificial Sequence
  Synthetic Primer
gagtccctggaccggattaaa

SEQ ID NO: 82 - DNA, 23 bp, Artificial Sequence
  Synthetic Primer
aatacatcacatagtgcctctgc

SEQ ID NO: 83 - DNA, 19 bp, Artificial Sequence
  Synthetic Primer
tgtcaggcaacggaatccc

SEQ ID NO: 84 - DNA, 19 bp, Artificial Sequence
  Synthetic Primer
caggtacggataggtgggc

SEQ ID NO: 85 - DNA, 20 bp, Artificial Sequence
  Synthetic Primer
gtccccactgtctacgaggt

SEQ ID NO: 86 - DNA, 19 bp, Artificial Sequence
  Synthetic Primer
cagacgacggggttggaag

SEQ ID NO: 87 - DNA, 22 bp, Artificial Sequence
  Synthetic Primer
agcatctagcagagatcaagcg

SEQ ID NO: 88 - DNA, 21 bp, Artificial Sequence
  Synthetic Primer
agccaacagacgaaaaagcag

SEQ ID NO: 89 - DNA, 19 bp, Artificial Sequence
  Synthetic Primer
ggtcctgcgtctgagaggt

SEQ ID NO: 90 - DNA, 21 bp, Artificial Sequence
  Synthetic Primer
ggccttcacattttcgatggt

SEQ ID NO: 91 - DNA, 22 bp, Artificial Sequence
  Synthetic Primer
ccaatggcctacaaaagaatgc

SEQ ID NO: 92 - DNA, 21 bp, Artificial Sequence
  Synthetic Primer
ttctgttgcgtctaactggca

SEQ ID NO: 93 - DNA, 20 bp, Artificial Sequence
  Synthetic Primer
ctctgcgtgcgcgttaaaag

SEQ ID NO: 94 - DNA, 23 bp, Artificial Sequence
  Synthetic Primer
caggcgactaatctcaaacatga

SEQ ID NO: 95 - DNA, 21 bp, Artificial Sequence
  Synthetic Primer
cctggcttccgcaacttacac

SEQ ID NO: 96 - DNA, 22 bp, Artificial Sequence
  Synthetic Primer
ggacttgtgcatgcggtactca

SEQ ID NO: 97 - DNA, 22 bp, Artificial Sequence
  Synthetic Primer
aaagatgtgcttcgagatgtgt

SEQ ID NO: 98 - DNA, 23 bp, Artificial Sequence
  Synthetic Primer
cactttgtcagttaccaacgtca

SEQ ID NO: 99 - DNA, 19 bp, Artificial Sequence
  Synthetic Primer
ccatgcaggtgagcttcgt

SEQ ID NO: 100 - DNA, 21 bp, Artificial Sequence
  Synthetic Primer
gaatctgcgagagacaccatc

SEQ ID NO: 101 - DNA, 23 bp, Artificial Sequence
  Synthetic Primer
cagtttgccccttttagtagtgc

SEQ ID NO: 102 - DNA, 20 bp, Artificial Sequence
  Synthetic Primer
ccagccgatactcgttcagc

SEQ ID NO: 103 - DNA, 25 bp, Artificial Sequence
  Synthetic Primer
tgctgtctccatgtttgatgtatct

SEQ ID NO: 104 - DNA, 22 bp, Artificial Sequence
  Synthetic Primer
tctctgctccccacctctaagt

SEQ ID NO: 105 - DNA, 23 bp, Artificial Sequence
  Synthetic Primer
tccagaaactaatggcagatccc

SEQ ID NO: 106 - DNA, 22 bp, Artificial Sequence
  Synthetic Primer
aattccctacgctttgggtttt

SEQ ID NO: 107 - DNA, 20 bp, Artificial Sequence
  Synthetic Primer
accccagggacagcatatca

SEQ ID NO: 108 - DNA, 20 bp, Artificial Sequence
  Synthetic Primer
tgcgatccgactcaccaata

SEQ ID NO: 109 - DNA, 23 bp, Artificial Sequence
  Synthetic Primer
cttgatggagaccctcctgtatg

SEQ ID NO: 110 - DNA, 21 bp, Artificial Sequence
  Synthetic Primer
agggtccaggtcctcttcaga

SEQ ID NO: 111 - DNA, 21 bp, Artificial Sequence
  Synthetic Primer
acagaaaggccctcaactgtc

SEQ ID NO: 112 - DNA, 22 bp, Artificial Sequence
  Synthetic Primer
tccttgatgtactcttggaccc

SEQ ID NO: 113 - DNA, 19 bp, Artificial Sequence
  Synthetic Primer
tgtccgtcagaacccatgc

SEQ ID NO: 114 - DNA, 21 bp, Artificial Sequence
  Synthetic Primer
aaagtcgaagttccatcgctc

SEQ ID NO: 115 - DNA, 19 bp, Artificial Sequence
  Synthetic Primer
gcggcgatcaagaagctgt

SEQ ID NO: 116 - DNA, 21 bp, Artificial Sequence
  Synthetic Primer
gcttggcgaagaaatcggaga

SEQ ID NO: 117 - DNA, 20 bp, Artificial Sequence
  Synthetic Primer
aacacagagaagcggatttc

SEQ ID NO: 118 - DNA, 20 bp, Artificial Sequence
  Synthetic Primer
aggtccagtcaaggatttca

SEQ ID NO: 119 - DNA, 20 bp, Artificial Sequence
  Synthetic Primer
atacaccgcttctgggtcag

SEQ ID NO: 120 - DNA, 20 bp, Artificial Sequence
  Synthetic Primer
tgcaagctttcttcatttcg

SEQ ID NO: 121 - DNA, 22 bp, Artificial Sequence
  Synthetic Primer
cagaagtcctacaaggtgtcca

SEQ ID NO: 122 - DNA, 21 bp, Artificial Sequence
  Synthetic Primer
ctctggttgaccgtaactgcg

SEQ ID NO: 123 - DNA, 20 bp, Artificial Sequence
  Synthetic Primer
agagctgcgttgcacttgtt

SEQ ID NO: 124 - DNA, 23 bp, Artificial Sequence
  Synthetic Primer
gcagtttaccaatcgttttgggg

SEQ ID NO: 125 - DNA, 23 bp, Artificial Sequence
  Synthetic Primer
acgtggatgattactacgacacc

SEQ ID NO: 126 - DNA, 21 bp, Artificial Sequence
  Synthetic Primer
tgcttttctcacggcatttct

SEQ ID NO: 127 - DNA, 20 bp, Artificial Sequence
  Synthetic Primer
atttaaagcaccagccatcg

SEQ ID NO: 128 - DNA, 20 bp, Artificial Sequence
  Synthetic Primer
tggaggcacaaggaggtatc

SEQ ID NO: 129 - DNA, 20 bp, Artificial Sequence
  Synthetic Primer
ggggagaagtcacgctatga

SEQ ID NO: 130 - DNA, 20 bp, Artificial Sequence
  Synthetic Primer
ctcagggcacaggaaaacat

SEQ ID NO: 131 - DNA, 20 bp, Artificial Sequence
  Synthetic Primer
cgcagctcaggaagaatgtg

SEQ ID NO: 132 - DNA, 21 bp, Artificial Sequence
  Synthetic Primer
tgaagtacactggcattgacg

SEQ ID NO: 133 - DNA, 21 bp, Artificial Sequence
  Synthetic Primer
tctggttcttacgtctgttgc

SEQ ID NO: 134 - DNA, 21 bp, Artificial Sequence
  Synthetic Primer
ctgtgcagtccctagctttcc

SEQ ID NO: 135 - DNA, 21 bp, Artificial Sequence
  Synthetic Primer
acacctatggaattgtcctgc

SEQ ID NO: 136 - DNA, 22 bp, Artificial Sequence
  Synthetic Primer
gaccagagagagctcagatacg

SEQ ID NO: 137 - DNA, 19 bp, Artificial Sequence
  Synthetic Primer
gtggcccggatgtgagaag

SEQ ID NO: 138 - DNA, 20 bp, Artificial Sequence
  Synthetic Primer
ggagcccttgtcggatgatg

SEQ ID NO: 139 - DNA, 19 bp, Artificial Sequence
  Synthetic Primer
tgacaacgacatcaacatc

SEQ ID NO: 140 - DNA, 19 bp, Artificial Sequence
  Synthetic Primer
gtgaccagagacaatgcag

SEQ ID NO: 141 - DNA, 19 bp, Artificial Sequence
  Synthetic Primer
gcccctcattaagcccaag

SEQ ID NO: 142 - DNA, 21 bp, Artificial Sequence
  Synthetic Primer
ttgtggtggtctgacagttcg

SEQ ID NO: 143 - DNA, 22 bp, Artificial Sequence
  Synthetic Primer
agacgctccctgtgatttatgt

SEQ ID NO: 144 - DNA, 20 bp, Artificial Sequence
  Synthetic Primer
ccgatggcagattccaaagg

SEQ ID NO: 145 - DNA, 20 bp, Artificial Sequence
  Synthetic Primer
ttgggctctatgggaaggac

SEQ ID NO: 146 - DNA, 21 bp, Artificial Sequence
  Synthetic Primer
gggagatgtatttgcagcgga

SEQ ID NO: 147 - DNA, 22 bp, Artificial Sequence
  Synthetic Primer
gctatcggggtaaagacctaca

SEQ ID NO: 148 - DNA, 21 bp, Artificial Sequence
  Synthetic Primer
cgtagcgtacctctggattgc

SEQ ID NO: 149 - DNA, 20 bp, Artificial Sequence
  Synthetic Primer
ctgctctacgacatgaacgg

SEQ ID NO: 150 - DNA, 22 bp, Artificial Sequence
  Synthetic Primer
gaaggtccctgatgtagtcgat

SEQ ID NO: 151 - DNA, 20 bp, Artificial Sequence
  Synthetic Primer
agtcccgtgaggtccgttag

SEQ ID NO: 152 - DNA, 23 bp, Artificial Sequence
  Synthetic Primer
agtcgttcatgttgtatagcagg

SEQ ID NO: 153 - DNA, 20 bp, Artificial Sequence
  Synthetic Primer
gagaggcactcagcttagcc

SEQ ID NO: 154 - DNA, 22 bp, Artificial Sequence
  Synthetic Primer
tccttttgtcgttggagatgac

SEQ ID NO: 155 - DNA, 21 bp, Artificial Sequence
  Synthetic Primer
tggagccgaacactctttact

SEQ ID NO: 156 - DNA, 23 bp, Artificial Sequence
  Synthetic Primer
cgggcaaaacataccagaagatg

SEQ ID NO: 157 - DNA, 19 bp, Artificial Sequence
  Synthetic Primer
gctcacctggcagcggaga

SEQ ID NO: 158 - DNA, 23 bp, Artificial Sequence
  Synthetic Primer
cgaccatttagcacctttgattt

SEQ ID NO: 159 - DNA, 18 bp, Artificial Sequence
  Synthetic Primer
tgcgcaagttcaccctca

SEQ ID NO: 160 - DNA, 24 bp, Artificial Sequence
  Synthetic Primer
tggacctcacacctaaggacaaag

SEQ ID NO: 161 - DNA, 26 bp, Artificial Sequence
  Synthetic Primer
ctgttgtcctacccaaataagactca

SEQ ID NO: 162 - DNA, 24 bp, Artificial Sequence
  Synthetic Primer
aattgccagatatgggaaagtttt

SEQ ID NO: 163 - DNA, 20 bp, Artificial Sequence
  Synthetic Primer
cccacactcaggtgatcgag

SEQ ID NO: 164 - DNA, 21 bp, Artificial Sequence
  Synthetic Primer
gagctgctttcgcttagtctt

SEQ ID NO: 165 - DNA, 22 bp, Artificial Sequence
  Synthetic Primer
ctcctgcaagagtttgatgtcc

SEQ ID NO: 166 - DNA, 21 bp, Artificial Sequence
  Synthetic Primer
tcatgccgatgtcatggtagg

SEQ ID NO: 167 - DNA, 24 bp, Artificial Sequence
  Synthetic Primer
acctggataaaaagaacccagaaa

SEQ ID NO: 168 - DNA, 24 bp, Artificial Sequence
  Synthetic Primer
ccttctccttcaccttcgtatcct

SEQ ID NO: 169 - DNA, 20 bp, Artificial Sequence
  Synthetic Primer
agaagctggcatcagaaaaa

SEQ ID NO: 170 - DNA, 20 bp, Artificial Sequence
  Synthetic Primer
ctggaaatgtgcaaaaacat

SEQ ID NO: 171 - DNA, 22 bp, Artificial Sequence
  Synthetic Primer
ggcaggttgtccttctattcag

SEQ ID NO: 172 - DNA, 19 bp, Artificial Sequence
  Synthetic Primer
cagtgtggggcgtaagagt

SEQ ID NO: 173 - DNA, 21 bp, Artificial Sequence
  Synthetic Primer
aggtccgaaaacactgtgagt

SEQ ID NO: 174 - DNA, 20 bp, Artificial Sequence
  Synthetic Primer
agcaagcggttcttcccttc

SEQ ID NO: 175 - DNA, 19 bp, Artificial Sequence
  Synthetic Primer
cccagcatctgcaaagctc

SEQ ID NO: 176 - DNA, 20 bp, Artificial Sequence
  Synthetic Primer
gtcaatgtacagctgccgca

SEQ ID NO: 177 - DNA, 19 bp, Artificial Sequence
  Synthetic Primer
ggccagatcctgtccaagc

SEQ ID NO: 178 - DNA, 21 bp, Artificial Sequence
  Synthetic Primer
gtgggtttccaccattagcac

SEQ ID NO: 179 - DNA, 23 bp, Artificial Sequence
  Synthetic Primer
gtagctctgatgagtgcaatgac

SEQ ID NO: 180 - DNA, 21 bp, Artificial Sequence
  Synthetic Primer
cagatatggcaactcccagtg

SEQ ID NO: 181 - DNA, 21 bp, Artificial Sequence
  Synthetic Primer
gagtccctggaccggattaaa

SEQ ID NO: 182 - DNA, 23 bp, Artificial Sequence
  Synthetic Primer
aatacatcacatagtgcctctgc

SEQ ID NO: 183 - DNA, 17 bp, Artificial Sequence
  Synthetic Primer
cgcgagctaagcaggag

SEQ ID NO: 184 - DNA, 20 bp, Artificial Sequence
  Synthetic Primer
gtccatagtcgctggaggag

SEQ ID NO: 185 - DNA, 19 bp, Artificial Sequence
  Synthetic Primer
ggtcctgcgtctgagaggt

SEQ ID NO: 186 - DNA, 21 bp, Artificial Sequence
  Synthetic Primer
ggccttcacattttcgatggt

SEQ ID NO: 187 - DNA, 20 bp, Artificial Sequence
  Synthetic Primer
ctctgcgtgcgcgttaaaag

SEQ ID NO: 188 - DNA, 23 bp, Artificial Sequence
  Synthetic Primer
caggcgactaatctcaaacatga

(19) United States
(12) Patent Application Publication
(10) Pub. No.: US 2017/0233792 A1
(43) Pub. Date: Aug. 17, 2017

(54) Fixation Method for Single Cell Analysis

(71) Applicants: Chun-Liang Chen, San Antonio, TX (US); Chun-Lin Lin, San Antonio, TX (US); Joseph Liu, San Antonio, TX (US); Tim Hui-Ming Huang, San Antonio, TX (US); Aaron Mark Horning, San Antonio, TX (US)

(72) Inventors: Chun-Liang Chen, San Antonio, TX (US); Chun-Lin Lin, San Antonio, TX (US); Joseph Liu, San Antonio, TX (US); Tim Hui-Ming Huang, San Antonio, TX (US); Aaron Mark Horning, San Antonio, TX (US)

(73) Assignee: THE BOARD OF REGENTS OF THE UNIVERSITY OF TEXAS SYSTEM, Austin, TX (US)

(21) Appl. No.: 15/411,025

(22) Filed: Jan. 20, 2017

Related U.S. Application Data
(60) Provisional application No. 62/281,012, filed on Jan. 20, 2016.

(51) Int. Cl. C12Q 1/68; G01N 1/30

(52) U.S. Cl. CPC C12Q 1/6806; G01N 1/30; G01N 2001/305

(57) ABSTRACT

A novel fixation method is presented that will circumvent both biological and safety concerns with current fixation methods.

### PRIORITY CLAIM

This Application claims priority to U.S. Provisional Patent application Ser. No. 62/281,012 filed Jan. 20, 2016, which is incorporated herein by reference in its entirety.

### BACKGROUND

Traditional methods of cell fixation for urine samples are problematic for both biological and safety reasons. Prior methods have resulted in genetic and protein cross-linking, thereby rendering the single cells nonviable for downstream analysis. Further, the previous fixation methods are chemically volatile and flammable, presenting a safety hazard.

Urine sample preparation is a key factor in obtaining adequate and representative cells for downstream analysis. Current methodology is usually a centrifugation for separation of tumor cells from urine based on their densities. However, this method often collects all types of cells and particles, and the recovery of the cells for subsequent molecular analysis is limited. Recent advances in size-selective microfiltration technology provide an alternative approach for isolation of tumor cells from urine sample.

Analysis of cells in the urine is often necessary in the diagnosis and treatment of urological cancers. In such analyses, cell morphology, protein expression and molecular alternations of the tumor cells are frequently targeted for detection. However, if urine is permitted to be collected at a clinical site and then transported to a central lab, the urine sample will often be stored at either room temperature or 4° C. for a prolonged period of hours before subsequent analysis can be completed. As a result, cells in urine will lyse or undergo apoptosis. In addition, the chemical composition of the urine sample is frequently altered upon standing as a result of environmental changes, for example, temperature change and protease digestion. Furthermore, bacterial contamination and other microorganisms may grow in urine, which may alter microfiltration and downstream analysis.

There remains a need for additional compositions and methods for preserving cells in the urine to enable proper analysis.

### SUMMARY

Fixation is needed for preserving the genetic material of cells for downstream analysis. The quality and integrity of cells of clinical samples are crucial for sensitivity and accuracy of clinical single cell analysis. In many occasions, the clinical samples may not be processed for single cell analysis immediately and the single cells would degrade dramatically. Traditional fixation methodologies would either induce cross-link between genetic materials and proteins affecting the downstream analysis or be chemically volatile and flammable. How to preserve the cells without compromising the downstream analysis and safety is an urgent need for biomedical research.

Certain embodiments are directed to fixation methods that circumvent both biological and safety concerns associated with current fixation methods. The method allows for a minimum 24 hour transport of single cells in urine that remain genetically stable for downstream analysis. The method can be used to preserve single cells collected for clinical analysis in other liquid samples and bodily fluids. Described herein is a fixation method for preserving single cells in a biological sample (e.g., a biological fluid such as urine, blood, or cerebrospinal fluid (CSF)) samples. In certain aspects the biological sample is a post digital rectal examination (DRE) urine sample. In a further aspect the sample is in condition for downstream analysis. The samples can be fixed upon collection without compromising the integrity of RNA or DNA (e.g., in exfoliated prostate cells (PSA/PSMA positive)) for at least 24 hours. This has been validated using microfluidic qRT-PCR with a prostate cancer 48-gene panel. The method can stabilize the genetic materials in the cells and provide a snapshot of molecular profiling of cells that may be subject to changes during the storage and transportation. The fixation method is not limited to urine and can be used to preserve single cells in other clinical liquid samples or body fluids for downstream single cell analysis.

The invention includes the fixation of exfoliated prostate cells in post DRE urine samples for downstream single cell analysis. The final 0.2% of paraformaldehyde was used to fix the cells in urine samples post DRE by adding an appropriate volume of 4% paraformaldehyde/PBS. In certain aspects the method is used to fix the cells in the urine samples collected after DRE. The cells are lightly fixed immediately when the urine is collected and the cell RNA and DNA quality will remain about the same as that at the point of fixation. In certain aspects the RNA and DNA quality is maintained for at least 12, 24, or 36 hours. In a particular aspect the RNA and DNA quality is maintained for at least 24 hours.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1B. The effects of urine on DU145 cell membrane permeability (trypan blue assay) and cell survival. (A) The cells were quantified using a Hemacytometer (Hausser Scientific, Cat #3120). (B) Cell survival of DUI45 in each treatment was shown as a ratio between live cells and total cells or percentage of live cells. Each data point was the average of triplicates.

FIGS. 2A-2C. (A) Urine degrades total RNA of LNCaP. (B) Urine degrades total RNA of PC3. (C) Urine degrades total RNA of BPH1.

FIG. 3. Paraformaldehyde (1%) fixation dramatically reduces gene detection using gene panel V.

FIG. 4 Decreased immunostaining of PSA and PSMA of BPH-1 fixed with 0.1% formaldehyde a-PSA.

FIG. 5. Decreased immunostaining of PSA and PSMA of PC3 fixed with 0.1% formaldehyde.

FIG. 6. Optimization of light fixation using 0.2-0.5% paraformaldehyde.

FIGS. 7A-7B. Gene expression of androgen up-regulated genes from fixed (0.2% paraformaldehyde) LNCaP cells is not different from that of unfixed LNCaP cells. (A) Network analysis was performed using Intelligent Pathway Analysis based on average gene expression. (B) Network analysis was performed using Intelligent Pathway Analysis based on average fold changes of gene expression (<0.5, >2).

FIG. 8. Gene expression of androgen up-regulated genes from fixed (0.2% paraformaldehyde) LNCaP cells is not different from that of unfixed LNCaP cells.

FIG. 9. Light fixation does not reduce gene detection in LNCaP cells.

### DESCRIPTION

Embodiments are directed to a fixative and methods for using the same in preparing a preserved sample or cells from a sample. In certain aspects a fixative comprises at least, at most, or about 2 to 5% paraformaldehyde, which is used to provide a final concentration of 0.2 to 0.5% in a fixed or preserved sample. In certain aspects the sample is a biological sample that comprises single cells. In a further aspect the biological sample is a urine sample, in particular a post digital rectal exam (DRE) urine sample. The fixative eliminates the need to immediately process urine samples. The fixative increases the stability of cells and maintains the integrity of nucleic acids in the cells, such as tumor cells, in urine or other biological samples. In further aspects the single cells can be isolated from the sample prior to being suspended in a solution with a final paraformaldehyde concentration of 0.2 to 0.5%. The cells can be separated from preserved urine samples using a number of different methods, including microfiltration, micromanipulation, and/or microinjection.

It will be appreciated given the description provided herein that the specific amounts of paraformaldehyde can vary in the stock solution and the fixative without departing from the excellent properties of the resulting fixative. The amount of paraformaldehyde in the stock solution may vary from between about 2 to about 20% (wt/vol). In certain aspects the stock solution may comprise 2 to 6% (wt/vol) paraformaldehyde. In particular aspects the stock solution can be about 4% paraformaldehyde. The fixed or preserved sample will have a final paraformaldehyde concentration of between 0.2 to 0.5% (wt/vol), including all values and ranges there between. In certain aspects the fixative will have a final paraformaldehyde concentration of about 0.2% (wt/vol).

The stock solution may be prepared by dissolving paraformaldehyde in deionized water. The pH of the reagent may be stabilized by adjusting it to between about 4.0 and about 10.0, including 4.0, 4.5, 5.0, 5.5, 6.0, 6.5, 7.0, 7.1, 7.2, 7.3, 7.4, 7.5, 7.6, 7.7, 7.8, 7.9, 8.0, 8.5, 9.0, 9.5 and 10.0. In a specific aspect, the pH is adjusted to about 7.4. pH stabilizing reagents for use in the reagents of the present invention include phosphate buffered saline (PBS), Tris-HCl, Hepes, citrate, and carbonate buffers, etc. In certain aspects the pH stabilizing reagent is PBS.

Upon mixing a stock solution of paraformaldehyde with a sample, such as urine, the final concentration of paraformaldehyde or fixative acts as a preservative that maintains the integrity of cells and the nucleic acids contained within the cells, i.e., decomposition of nucleic acids such as DNA, RNA and microRNA in the cells is inhibited or reduced. Typically, the stock paraformaldehyde solution is mixed with a sample within about 10, 9, 8, 7, 6, 5, 4, 3, 2 or 1 hour of collecting the sample from a subject, or within about 60, 55, 50, 45, 40, 35, 30, 25, 20, 15, 10, 9, 8, 7, 6, 5, 4, 3, 2 or 1 minutes of collecting the sample from the subject. In particular aspects, the stock paraformaldehyde solution is mixed with the sample within about 3 hours, and preferably within 20 minutes, of collecting the sample from the subject.

Upon mixing the stock paraformaldehyde solution with the sample, forming a fixed or preserved sample, the fixed or preserved sample may be stored for a week at 4° C. until analyzed. For long term of storage, the stock can be frozen at −80° C. Alternatively, the fixed sample may be stored at room temperature or stored at a temperature between 0° C. and room temperature, such as at 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., or higher.

Many methods can be used for isolation of cells from the fixed sample. Centrifugation is one of the most common methods and involves use of centrifugal force for sedimentation of heterogeneous mixtures in the fixed sample. However, the pelleted materials at the bottom of centrifuge tube could contain not only cells, but also contaminating protein precipitates, cellular materials, red blood cells, and bacteria, etc.

Another method is to use antibodies or aptamers against the markers on the surface of cells to improve the purity of the collected material. Antibodies and aptamers can be attached to a solid support such as magnetic beads, ferrofluids, surfaces of microfluidic channels, etc. Magnetic beads or ferrofluids, coated with antibodies that recognize the cell surface marker of interest, can be mixed with fixed sample or a pellet collected from fixed sample. The cells will be captured on the surfaces of the magnetic beads or ferrofluids during incubation. Magnets can be used to collect the magnetic beads and ferrofluids and collect the cells of interest. If the antibodies or aptamers are coated on the surface of a microfluidic chip, the cells of interest will be captured on the surface of the microfluidic chip when the fixed sample flows through the chip.

Another method of affinity capture is to incubate the fixed sample or pellet from the fixed sample with antibody (antibodies) and/or aptamers, specific for cell surface markers of interest, that are conjugated with either avidin or biotin. After incubation, the cells can be collected on surfaces coated with biotin or avidin, respectively, to form biotin/avidin pairs. The surfaces can be magnetic beads, ferrofluids, microfluidic chips, etc.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

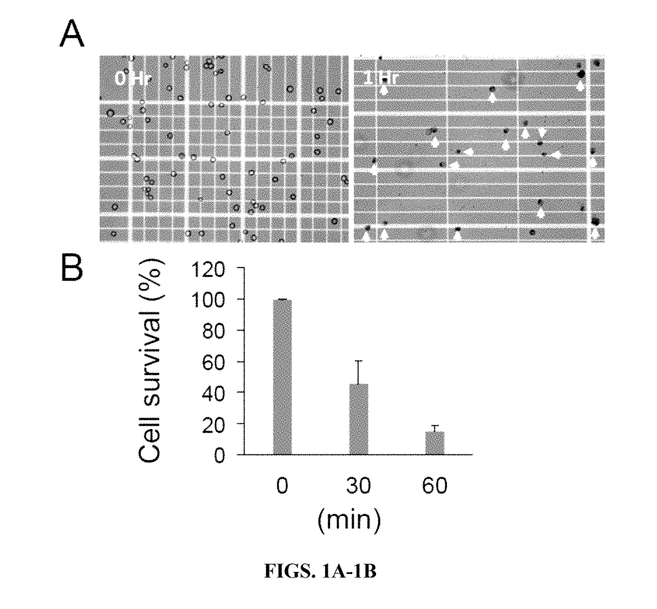

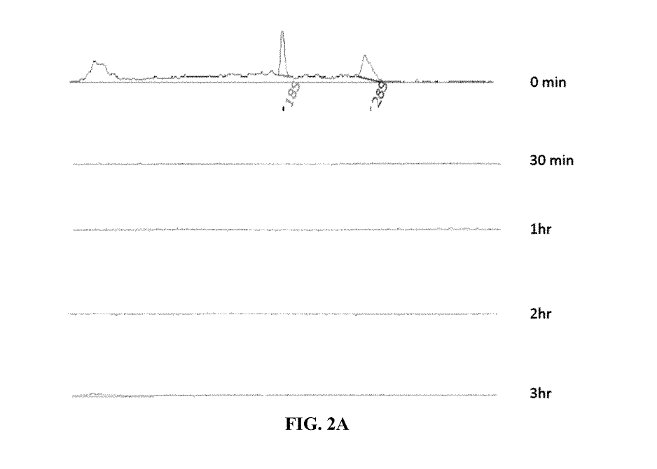

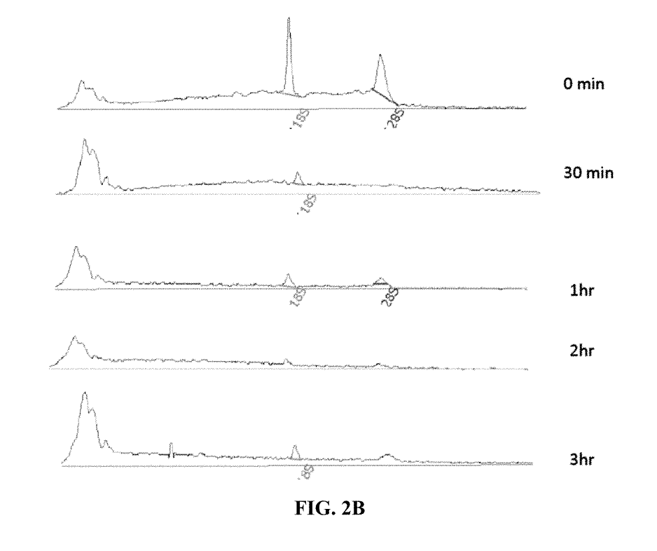

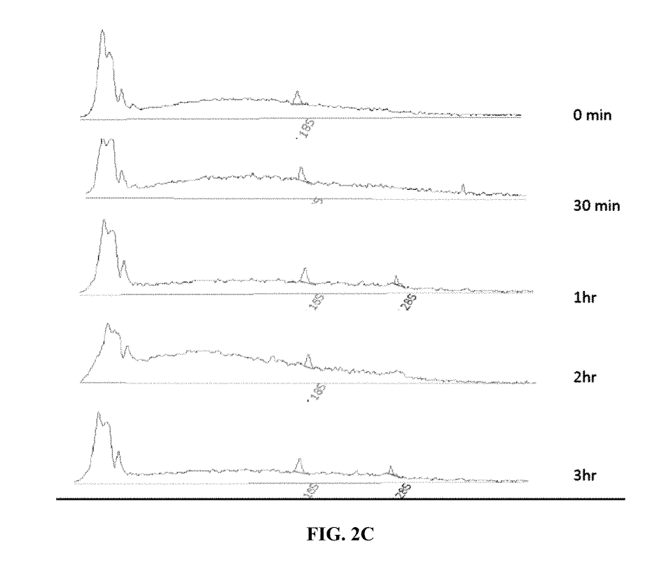

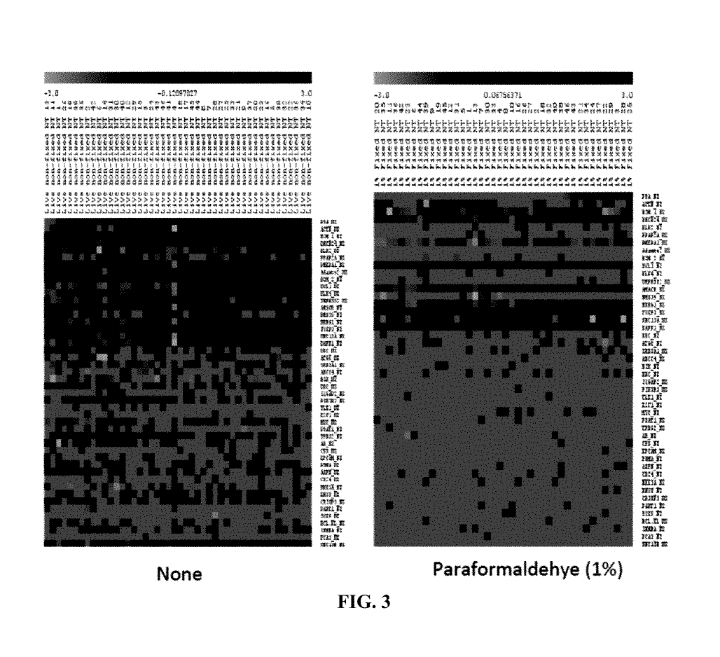

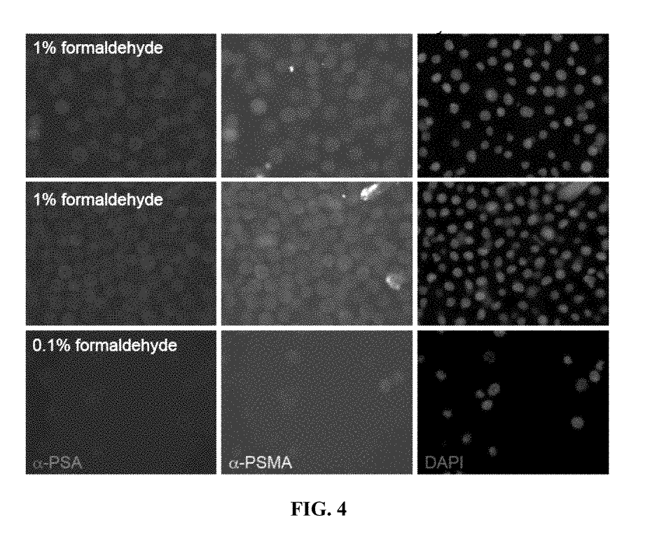

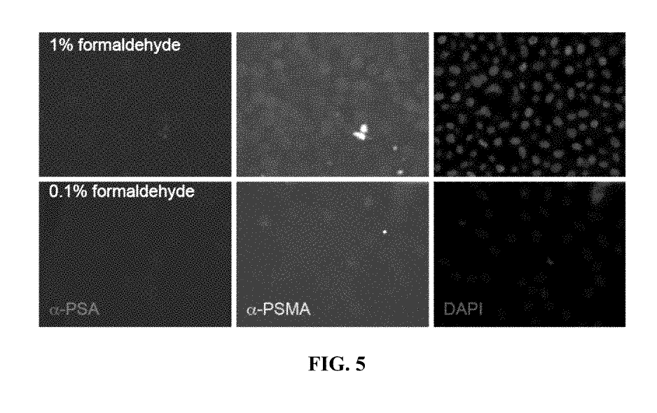

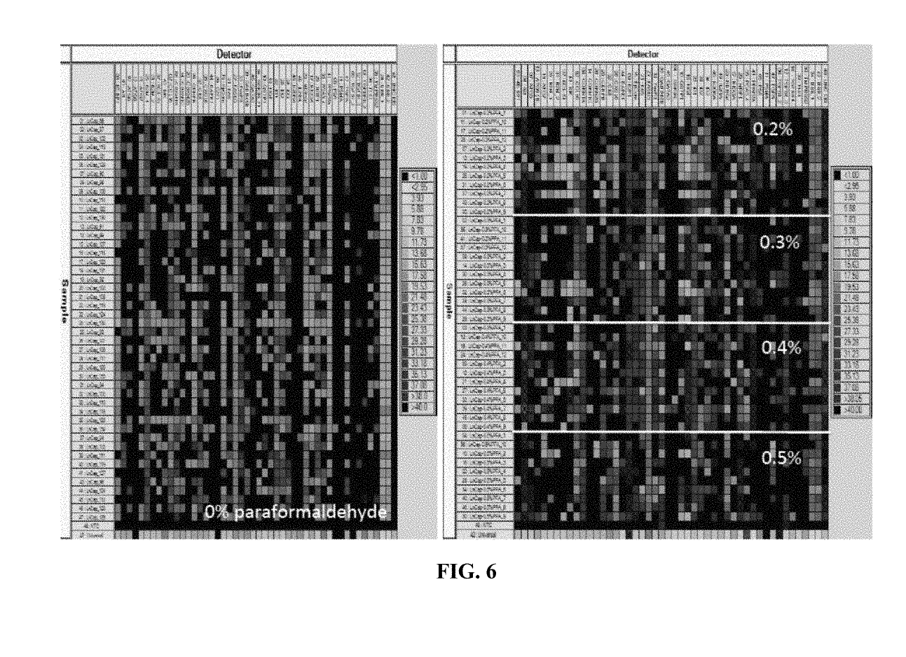

Trypan blue cell survival assay. DU145 cells (ATCC) were cultured in vented-caps flasks (Corning, 430720U) in media RPMI 1640 (Gibco) supplemented with 10% FBS (Gibco) and 1% Penecillin/Streptomycin (Gibco) at 37° C. in an Incubator fanned with 5% CO₂. DU145 cells were harvested with 0.05% trypsin. About 50,000 cells were spiked in 1.5 ml fresh human urine in centrifuge tubes for 0 min, 30 min, and 60 min at room temperature. DU145 cells after urine incubation were separated from urine using centrifugation at 500×g for 5 min. The cell pellets were washed with PBS buffer twice and then stained with 0.2% trypan blue (Sigma-Aldrich, T8154) for cell survival assay. The cells without staining (trypan blue negative) were live cells and the cells stained blue were permeated dead cells. The cells were quantified using a Hemacytometer (Hausser Scientific, Cat #3120) (See, FIG. 1A). Cell survival of DU145 in each treatment was shown as a ratio between live cells and total cells or percentage of live cells. Each data point was the average of triplicates (See, FIG. 1B).

Evaluation of cell RNA integrity. PC3, LNCaP, and BPH-1 prostate cancer cell lines were grown and harvested as previously described. About 0.5 million cells were spiked into 1.5 ml human urine at room temperature as above for 0 minute, 30 minutes, 1 hour, 2 hours, or 3 hours. The cells were centrifuged at 500×g for 5 minutes for collection and washed twice with 1 ml PBS. The cell pellets were lysed in Trizol LS reagent (Invitrogen, 10296-010) and RNA was isolated using RNAeasy mini kit (Qiagen, 74104) following the manufacturer's protocol. RNA (˜1 ng) integrity was evaluated using Agilent 2100 Bioanalyzer (Agilent Technlogies, G2939AA) according to the manufacturer's protocol.

Isolation of urine single cells and cultured prostate cancer cell lines. LNCaP cells (ATCC) were cultured in vented-caps flasks (Corning, 430720U) in media RPMI 1640 (Gibco) supplemented with 10% FBS (Gibco) and 1% Penicillin/Streptomycin (Gibco) at 37° C. in an Incubator fanned with 5% CO₂ Cells were trypsinized in Trypsin LE (Gibco), collected, centrifuged at 400×g and washed once in 1× PBS before being fixed in 0, 0.2, 0.3, 0.4, 0.5 or 1% paraformaldehyde for 20 minutes, 1 hour, or up to 24 hours at room temperature. For prostate cells in post-DRE urine samples, the paraformaldehyde was added according to the final concentrations for either 1 hour or 24 hours. Following fixation, the cells were centrifuged at 700×g (because of their increased buoyancy post-fixation, it was necessary to increase the centrifugation speed) then washed in 1×PBS two times. To prevent cells adhering to the plastic petri dishes during single-cell picking, cells were suspended in a 1:1 mix of 1× PBS and full culture media (described above) because the FBS prevents electrostatic “stickiness” between the cells and petri dish. Single cells were picked with a micromanipulator tip (Origio, MXL3-50) and ejected into a strip-PCR tube (Genessee, 27-125UA) with 4 μl of 0.4% Triton-X in 2× Reaction Mix (ThermoFisher Scientific, 11753-100) while keeping the samples at 4° C. after ejecting into the tube. Single-cell samples were frozen at −80° C. until reverse transcription and pre-amplification.

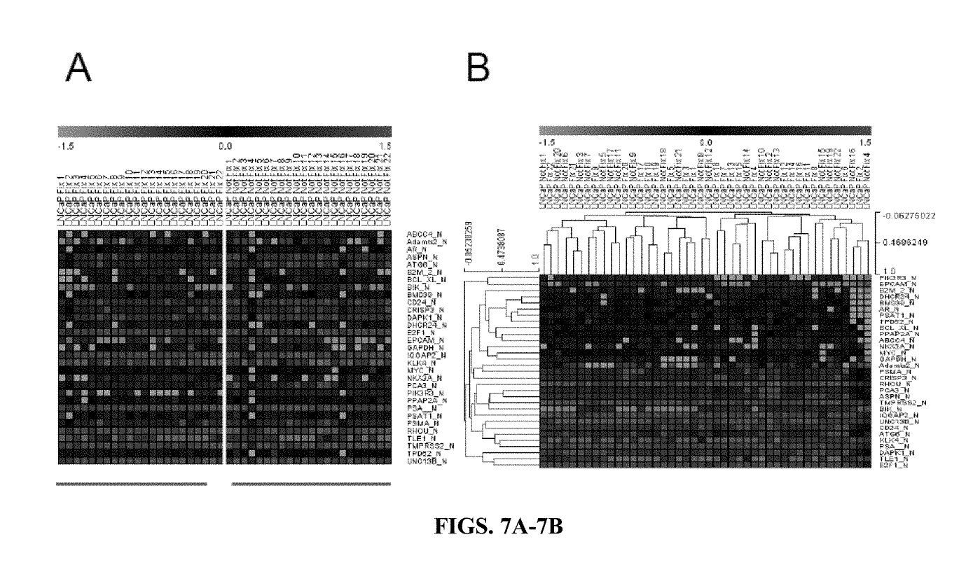

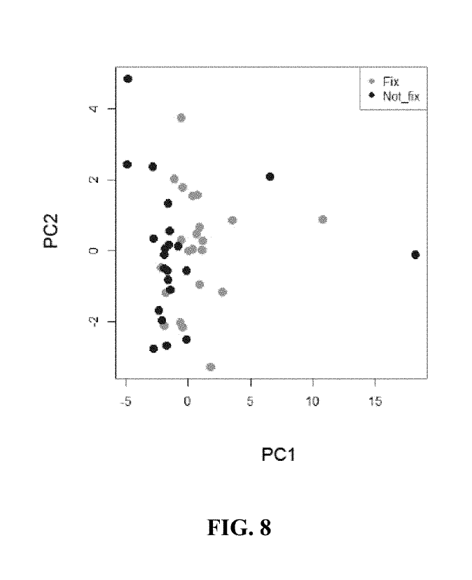

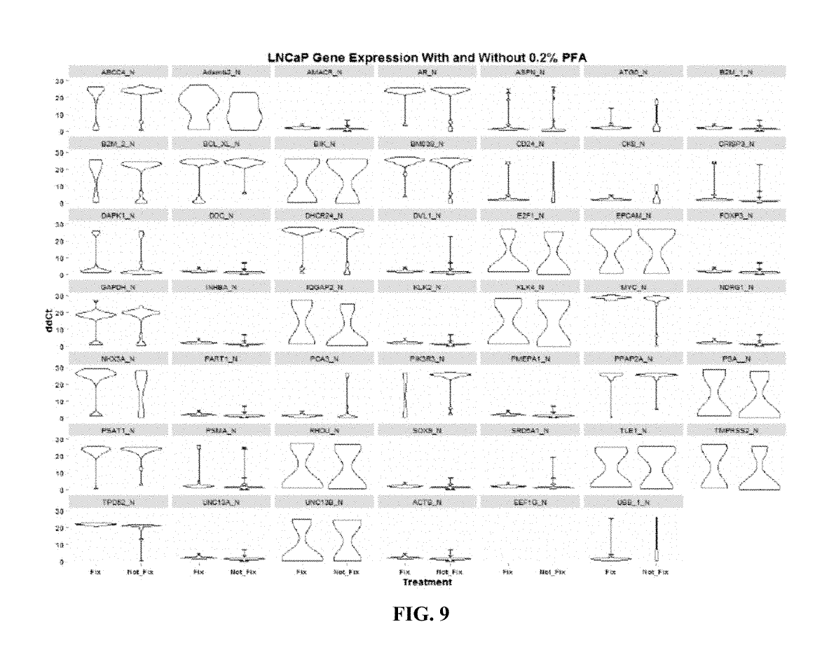

Single cell qRT-PCR using BioMark™ system. Single cultured prostate cancer cells or urine prostate cells isolated and stored in 2× reaction buffer were subject to single cell qRT-PCR using BioMark™ system following previously described. The gene panels used for gene expression profiling are androgen-upregulated gene panel V (Table 1), gene panel VII (Table 2). The gene expression levels were determined and all Ct values were compared to the expression of the housekeeping gene, UBB. Relative gene expression is based on negative delta delta Ct values (−ΔΔCt), which were calculated from a gene's max dCt value. Unsupervised hierarchical clustering and Principle Component Analysis (PCA) were performed in MeV. Network analysis was performed using Intelligent Pathway Analysis based on average gene expression (see, FIG. 7A) and/or average fold changes of gene expression (<0.5, >2) (see, FIG. 7B). Statistical analysis was carried out using Student t test with p<0.05 considered as significant.

Immunostaining. Dissociated cultured prostate cancer cells (˜0.5 million), BPH-1 and PC3, were fixed in 0.2% and 1% paraformaldehyde/PBS for 20 minutes and washed with PBS twice. Cell pellets were suspended in 100 μl PBS+5% FBS+0.2% tween 20 and cells were labeled with polyclonal rabbit α-PSA (1:100, Dako, #A0562), α-hPSMA/FOLH1-APC (1:10, R&D system, #FAB4234A) and incubated on ice for 15 min with light proof (aluminum foil). Cells were centrifuged in a bench microcentrifuge for 20 seconds. The cell pellets were washed 1 ml PBS+5% FBS+0.2% tween 20 to remove the primary antibodies. A secondary antibody (α-rabbit IgG-Cy3) was diluted (v:v=1:500) in 100 μl PBS+5% FBS+0.2% tween 20+0.5 μg/ml DAPI at RT for 15 minutes. The cells were washed with 100 μl PBS+5% FBS+0.2% tween 20 to removed the antibodies and removed into 6 well culture plate for fluorescent image analysis. The staining of PSA, FOLH1, and DAPI is located in cytoplasm, membrane, and nuclei, respectively. The images of immunostaining were taken using an inverted fluorescent microscope EVOS fl (Advanced Microscopy Group).

TABLE 1
Gene panel V (androgen-upregulated genes)

| Gene ID | FORWARD (5′-3′) | REVERSE (5′-3′) |
|---|---|---|
| ABCC4 | AGCTGAGAATGACGCACAGAA | ATATGGGCTGGATTACTT |
| | (SEQ ID NO: 1) | TGGC |
| | | (SEQ ID NO: 2) |
| ACTB | CATGTACGTTGCTATCCAGGC | CTCCTTAATGTCACGCAC |
| | (SEQ ID NO: 3) | GAT |
| | | (SEQ ID NO: 4) |
| ADAMTS2 | GTGCATGTGGTGTATCGCC | AGGACCTCGATGTTGTAG |
| | (SEQ ID NO: 5) | TCA |
| | | (SEQ ID NO: 6) |
| AMACR | TCAACTATTTGGCTTTGTCAGG | GTGAGAATCCGTATGCCC |
| | (SEQ ID NO: 7) | C |
| | | (SEQ ID NO: 8) |
| AR | TCCATCTTGTCGTCTTCGGAA | GGGCTGGTTGTTGTCGTG |
| | (SEQ ID NO: 9) | T |
| | | (SEQ ID NO: 10) |
| ASPN | CTCTGCCAAACCCTTCTTTAGC | CGTGAATAGCACTGACAT |
| | (SEQ ID NO: 11) | CCAA |
| | | (SEQ ID NO: 12) |
| ATG6 | CCATGCAGGTGAGCTTCGT | GAATCTGCGAGAGACACC |
| (BECN1) | (SEQ ID NO: 13) | ATC |
| | | (SEQ ID NO: 14) |
| BCL-XL | ACCCCAGGGACAGCATATCA | TGCGATCCGACTCACCAA |
| | (SEQ ID NO: 15) | TA |
| | | (SEQ ID NO: 16) |
| BIK | CTTGATGGAGACCCTCCTGTATG | AGGGTCCAGGTCCTCTTC |
| | (SEQ ID NO: 17) | AGA |
| | | (SEQ ID NO: 18) |
| B2M_1* | TGCTGTCTCCATGTTTGATGTATCT | TCTCTGCTCCCCACCTCT |
| | (SEQ ID NO: 19) | AAGT |
| | | (SEQ ID NO: 20) |
| B2M_2* | TCCAGAAACTAATGGCAGATCCC | AATTCCCTACGCTTTGGG |
| | (SEQ ID NO: 21) | TTTT |
| | | (SEQ ID NO: 22) |
| BM039 | TGAACTGACAACAATCCTGAAGG | CTTGCACGCTTTTCCTCA |
| | (SEQ ID NO: 23) | CAC |
| | | (SEQ ID NO: 24) |
| CK8 | CAGAAGTCCTACAAGGTGTCCA | CTCTGGTTGACCGTAACT |
| | (SEQ ID NO: 25) | GCG |
| | | (SEQ ID NO: 26) |
| CD24 | CTCCTACCCACGCAGATTTATTC | AGAGTGAGACCACGAAGA |
| | (SEQ ID NO: 27) | GAC |
| | | (SEQ ID NO: 28) |
| CRISP3 | TACAGACACAGTAACCCAAAGGA | TGGATTGCTTGTGACCAT |
| | (SEQ ID NO: 29) | GAG |
| | | (SEQ ID NO: 30) |
| DAPK1 | ACGTGGATGATTACTACGACACC | TGCTTTTCTCACGGCATT |
| | (SEQ ID NO: 31) | TCT |
| | | (SEQ ID NO: 32) |
| DDC | TGGGGACCACAACATGCTG | TCAGGGCAGATGAATGCA |
| | (SEQ ID NO: 33) | CTG |
| | | (SEQ ID NO: 34) |
| DHCR24 | CACTGTCTCACTACGTGTCGG | CCAGCCAATGGAGGTCAG |
| | (SEQ ID NO: 35) | C |
| | | (SEQ ID NO: 36) |
| DVL1 | GCGGGAGATCGTTTCCCAG | CGGCGCTCATGTCACTCT |
| | (SEQ ID NO: 37) | T |
| | | (SEQ ID NO: 38) |
| E2F1 | ACGCTATGAGACCTCACTGAA | TCCTGGGTCAACCCCTCA |
| | (SEQ ID NO: 39) | AG |
| | | (SEQ ID NO: 40) |
| EPCAM | AATCGTCAATGCCAGTGTACTT | TCTCATCGCAGTCAGGAT |
| | (SEQ ID NO: 41) | CATAA |
| | | (SEQ ID NO: 42) |
| FOXP3 | GTGGCCCGGATGTGAGAAG | GGAGCCCTTGTCGGATGA |
| | (SEQ ID NO: 43) | TG |
| | | (SEQ ID NO: 44) |
| GAPDH | ACAACTTTGGTATCGTGGAAGG | GCCATCACGCCACAGTTT |
| | (SEQ ID NO: 45) | C |
| | | (SEQ ID NO: 46) |
| INHBA | CCTCCCAAAGGATGTACCCAA | CTCTATCTCCACATACCC |
| | (SEQ ID NO: 47) | GTTCT |
| | | (SEQ ID NO: 48) |
| IQGAP2 | AGACCCCGCTATGGCTCTATT | GCTTCCTCTAAGTGGCAC |
| | (SEQ ID NO: 49) | AGAT |
| | | (SEQ ID NO: 50) |
| KLK2 | TCAGAGCCTGCCAAGATCAC | CACAAGTGTCTTTACCAC |
| | (SEQ ID NO: 51) | CTGT |
| | | (SEQ ID NO: 52) |
| KLK4 | GCCAAATCATAAACGGCGAGG | CGCCCGAGCAGAACAATT |
| | (SEQ ID NO: 53) | C |
| | | (SEQ ID NO: 54) |
| MYC | GGCTCCTGGCAAAAGGTCA | CTGCGTAGTTGTGCTGAT |
| | (SEQ ID NO: 55) | GT |
| | | (SEQ ID NO: 56) |
| NDRG1 | CTCCTGCAAGAGTTTGATGTCC | TCATGCCGATGTCATGGT |
| | (SEQ ID NO: 57) | AGG |
| | | (SEQ ID NO: 58) |
| NKX3A | CCCACACTCAGGTGATCGAG | GAGCTGCTTTCGCTTAGT |
| | (SEQ ID NO: 59) | CTT |
| | | (SEQ ID NO: 60) |
| PART1 | AAGGCCGTGTCAGAACTCAA | GTTTTCCATCTCAGCCTG |
| | (SEQ ID NO: 61) | GA |
| | | (SEQ ID NO: 62) |
| PCA3 | GCACATTTCCAGCCCCTTTAAA | GGGCGAGGCTCATCGAT |
| | (SEQ ID NO: 63) | (SEQ ID NO: 64) |
| PIK3R3 | TACAATACGGTGTGGAGTATGGA | TCATTGGCTTAGGTGGCT |
| | (SEQ ID NO: 65) | TTG |
| | | (SEQ ID NO: 66) |
| PPAP2A | GGCAGGTTGTCCTTCTATTCAG | CAGTGTGGGGCGTAAGAG |
| | (SEQ ID NO: 67) | T |
| | | (SEQ ID NO: 68) |
| PSA_ | GTGTGTGGACCTCCATGTTATT | TGCCCCATGACGTGATAC |
| | (SEQ ID NO: 69) | CT |
| | | (SEQ ID NO: 70) |
| PSAT1 | TGCCGCACTCAGTGTTGTTAG | GCAATTCCCGCACAAGAT |
| | (SEQ ID NO: 71) | TCT |
| | | (SEQ ID NO: 72) |
| PSMA | CGGAGCAAACCTCGGAGTC | GCGGCCAGAAACAATGGA |
| | (SEQ ID NO: 73) | TAG |
| | | (SEQ ID NO: 74) |
| RHOU | GCTACCCCACCGAGTACATC | GGCTCACGACACTGAAGC |
| | (SEQ ID NO: 75) | A |
| | | (SEQ ID NO: 76) |
| S0X9 | AGCGAACGCACATCAAGAC | CTGTAGGCGATCTGTTGG |
| | (SEQ ID NO: 77) | GG |
| | | (SEQ ID NO: 78) |
| SRD5A1 | TCAGACGAACTCAGTGTACGG | CGTAGTGGACGAGGAACA |
| | (SEQ ID NO: 79) | TGG |
| | | (SEQ ID NO: 80) |
| TLE1 | GAGTCCCTGGACCGGATTAAA | AATACATCACATAGTGCC |
| | (SEQ ID NO: 81) | TCTGC |
| | | (SEQ ID NO: 82) |
| PMEPA1 | TGTCAGGCAACGGAATCCC | CAGGTACGGATAGGTGGG |
| | (SEQ ID NO: 83) | C |
| | | (SEQ ID NO: 84) |
| TMPRSS2 | GTCCCCACTGTCTACGAGGT | CAGACGACGGGGTTGGAA |
| | (SEQ ID NO: 85) | G |
| | | (SEQ ID NO: 86) |
| TPD52 | AGCATCTAGCAGAGATCAAGCG | AGCCAACAGACGAAAAAG |
| | (SEQ ID NO: 87) | CAG |
| | | (SEQ ID NO: 88) |
| UBB_1* | GGTCCTGCGTCTGAGAGGT | GGCCTTCACATTTTCGAT |
| | (SEQ ID NO: 89) | GGT |
| | | (SEQ ID NO: 90) |
| UBB_2* | GGTCCTGCGTCTGAGAGGT | GGCCTTCACATTTTCGAT |
| | (SEQ ID NO: 89) | GGT |
| | | (SEQ ID NO: 90) |
| UNC13A | CCAATGGCCTACAAAAGAATGC | TTCTGTTGCGTCTAACTG |
| | (SEQ ID NO: 91) | GCA |
| | | (SEQ ID NO: 92) |
| UNC13B | CTCTGCGTGCGCGTTAAAAG | CAGGCGACTAATCTCAAA |
| | (SEQ ID NO: 93) | CATGA |
| | | (SEQ ID NO: 94) |

TABLE 2
Gene Panel VII

| Gene ID | FORWARD (5′-3′) | REVERSE (5′-3′) |
|---|---|---|
| AR | CCTGGCTTCCGCAACTTACAC | GGACTTGTGCATGCGGTACT |
| | (SEQ ID NO: 95) | CA |
| | | (SEQ ID NO: 96) |
| ATG5 | AAAGATGTGCTTCGAGATGTGT | CACTTTGTCAGTTACCAACG |
| | (SEQ ID NO: 97) | TCA |
| | | (SEQ ID NO: 98) |
| ATG6 | CCATGCAGGTGAGCTTCGT | GAATCTGCGAGAGACACCAT |
| (BECN1) | (SEQ ID NO: 99) | C |
| | | (SEQ ID NO: 100) |
| ATG7 | CAGTTTGCCCCTTTTAGTAGTG | CCAGCCGATACTCGTTCAGC |
| | C | (SEQ ID NO: 102) |
| | (SEQ ID NO: 101) | |
| B2M_1 | TGCTGTCTCCATGTTTGATGTA | TCTCTGCTCCCCACCTCTAA |
| | TCT | GT |
| | (SEQ ID NO: 103) | (SEQ ID NO: 104) |
| B2M_2 | TCCAGAAACTAATGGCAGATCC | AATTCCCTACGCTTTGGGTT |
| | C | TT |
| | (SEQ ID NO: 105) | (SEQ ID NO: 106) |
| BCL-XL | ACCCCAGGGACAGCATATCA | TGCGATCCGACTCACCAATA |
| | (SEQ ID NO: 107) | (SEQ ID NO: 108) |
| BIK | CTTGATGGAGACCCTCCTGTA | AGGGTCCAGGTCCTCTTCAG |
| | TG | A |
| | (SEQ ID NO: 109) | (SEQ ID NO: 110) |
| CCL16 | ACAGAAAGGCCCTCAACTGTC | TCCTTGATGTACTCTTGGAC |
| | (SEQ ID NO: 111) | CC |
| | | (SEQ ID NO: 112) |
| CDKN1A | TGTCCGTCAGAACCCATGC | AAAGTCGAAGTTCCATCGCT |
| | (SEQ ID NO: 113) | C |
| | | (SEQ ID NO: 114) |
| CDKN1C | GCGGCGATCAAGAAGCTGT | GCTTGGCGAAGAAATCGGAG |
| | (SEQ ID NO: 115) | A |
| | | (SEQ ID NO: 116) |
| CDKN2B | AACACAGAGAAGCGGATTTC | AGGTCCAGTCAAGGATTTCA |
| | (SEQ ID NO: 117) | (SEQ ID NO: 118) |
| CENPN | ATACACCGCTTCTGGGTCAG | TGCAAGCTTTCTTCATTTCG |
| | (SEQ ID NO: 119) | (SEQ ID NO: 120) |
| CK8 | CAGAAGTCCTACAAGGTGTCCA | CTCTGGTTGACCGTAACTGC |
| | (SEQ ID NO: 121) | G |
| | | (SEQ ID NO: 122) |
| CXCL6 | AGAGCTGCGTTGCACTTGTT | GCAGTTTACCAATCGTTTTG |
| | (SEQ ID NO: 123) | GGG |
| | | (SEQ ID NO: 124) |
| DAPK1 | ACGTGGATGATTACTACGACA | TGCTTTTCTCACGGCATTTC |
| | CC | T |
| | (SEQ ID NO: 125) | (SEQ ID NO: 126) |
| 4E-BP1 | ATTTAAAGCACCAGCCATCG | TGGAGGCACAAGGAGGTATC |
| | (SEQ ID NO: 127) | (SEQ ID NO: 128) |
| E2F1 | GGGGAGAAGTCACGCTATGA | CTCAGGGCACAGGAAAACAT |
| | (SEQ ID NO: 129) | (SEQ ID NO: 130) |
| EpCam | CGCAGCTCAGGAAGAATGTG | TGAAGTACACTGGCATTGAC |
| | (SEQ ID NO: 131) | G |
| | | (SEQ ID NO: 132) |
| FAS | TCTGGTTCTTACGTCTGTTGC | CTGTGCAGTCCCTAGCTTTC |
| | (SEQ ID NO: 133) | C |
| | | (SEQ ID NO: 134) |
| FASLG | ACACCTATGGAATTGTCCTGC | GACCAGAGAGAGCTCAGATA |
| | (SEQ ID NO: 135) | CG |
| | | (SEQ ID NO: 136) |
| FoxP3 | GTGGCCCGGATGTGAGAAG | GGAGCCCTTGTCGGATGATG |
| | (SEQ ID NO: 137) | (SEQ ID NO: 138) |
| GADD45B | TGACAACGACATCAACATC | GTGACCAGAGACAATGCAG |
| | (SEQ ID NO: 139) | (SEQ ID NO: 140) |
| GATA3 | GCCCCTCATTAAGCCCAAG | TTGTGGTGGTCTGACAGTTC |
| | (SEQ ID NO: 141) | G |
| | | (SEQ ID NO: 142) |
| GSK3B | AGACGCTCCCTGTGATTTATGT | CCGATGGCAGATTCCAAAGG |
| | (SEQ ID NO: 143) | (SEQ ID NO: 144) |
| GSTP1 | TTGGGCTCTATGGGAAGGAC | GGGAGATGTATTTGCAGCGG |
| | (SEQ ID NO: 145) | A |
| | | (SEQ ID NO: 146) |
| HGF | GCTATCGGGGTAAAGACCTACA | CGTAGCGTACCTCTGGATTG |
| | (SEQ ID NO: 147) | C |
| | | (SEQ ID NO: 148) |
| ID1 | CTGCTCTACGACATGAACGG | GAAGGTCCCTGATGTAGTCG |
| | (SEQ ID NO: 149) | AT |
| | | (SEQ ID NO: 150) |
| ID2 | AGTCCCGTGAGGTCCGTTAG | AGTCGTTCATGTTGTATAGC |
| | (SEQ ID NO: 151) | AGG |
| | | (SEQ ID NO: 152) |
| ID3 | GAGAGGCACTCAGCTTAGCC | TCCTTTTGTCGTTGGAGATG |
| | (SEQ ID NO: 153) | AC |
| | | (SEQ ID NO: 154) |
| IL20RA | TGGAGCCGAACACTCTTTACT | CGGGCAAAACATACCAGAAG |
| | (SEQ ID NO: 155) | ATG |
| | | (SEQ ID NO: 156) |
| IL2RA | GCTCACCTGGCAGCGGAGA | CGACCATTTAGCACCTTTGA |
| | (SEQ ID NO: 157) | TTT |
| | | (SEQ ID NO: 158) |
| PSA | TGCGCAAGTTCACCCTCA | TGGACCTCACACCTAAGGAC |
| | (SEQ ID NO: 159) | AAAG |
| | | (SEQ ID NO: 160) |
| PSMA | CTGTTGTCCTACCCAAATAAGA | AATTGCCAGATATGGGAAAG |
| | CTCA | TTTT |
| | (SEQ ID NO: 161) | (SEQ ID NO: 162) |
| NKX3A | CCCACACTCAGGTGATCGAG | GAGCTGCTTTCGCTTAGTCT |
| | (SEQ ID NO: 163) | T |
| | | (SEQ ID NO: 164) |
| NDRG1 | CTCCTGCAAGAGTTTGATGTCC | TCATGCCGATGTCATGGTAG |
| | (SEQ ID NO: 165) | G |
| | | (SEQ ID NO: 166) |
| NRP1 | ACCTGGATAAAAAGAACCCAGA | CCTTCTCCTTCACCTTCGTA |
| | AA | TCCT |
| | (SEQ ID NO: 167) | (SEQ ID NO: 168) |
| PCA3 | AGAAGCTGGCATCAGAAAAA | CTGGAAATGTGCAAAAACAT |
| | (SEQ ID NO: 169) | (SEQ ID NO: 170) |
| PPAP2A | GGCAGGTTGTCCTTCTATTCAG | CAGTGTGGGGCGTAAGAGT |
| | (SEQ ID NO: 171) | (SEQ ID NO: 172) |
| TGFA | AGGTCCGAAAACACTGTGAGT | AGCAAGCGGTTCTTCCCTTC |
| | (SEQ ID NO: 173) | (SEQ ID NO: 174) |
| TGF- | CCCAGCATCTGCAAAGCTC | GTCAATGTACAGCTGCCGCA |
| B1_2 | (SEQ ID NO: 175) | (SEQ ID NO: 176) |
| TGFB1_3 | GGCCAGATCCTGTCCAAGC | GTGGGTTTCCACCATTAGCA |
| | (SEQ ID NO: 177) | C |
| | | (SEQ ID NO: 178) |
| TGFBRII | GTAGCTCTGATGAGTGCAATG | CAGATATGGCAACTCCCAGT |
| | AC | G |
| | (SEQ ID NO: 179) | (SEQ ID NO: 180) |
| TLE1 | GAGTCCCTGGACCGGATTAAA | AATACATCACATAGTGCCTC |
| | (SEQ ID NO: 181) | TGC |
| | | (SEQ ID NO: 182) |
| TMPRSS2 | CGCGAGCTAAGCAGGAG | GTCCATAGTCGCTGGAGGAG |
| | (SEQ ID NO: 183) | (SEQ ID NO: 184) |
| UBB_1 | GGTCCTGCGTCTGAGAGGT | GGCCTTCACATTTTCGATGG |
| | (SEQ ID NO: 185) | T |
| | | (SEQ ID NO: 186) |
| UNC13B | CTCTGCGTGCGCGTTAAAAG | CAGGCGACTAATCTCAAACA |
| F | (SEQ ID NO: 187) | TGA |
| | | (SEQ ID NO: 188) |

1. A method of measuring gene expression levels of cells isolated from a biological sample comprising:
isolating a cell dispersion from a biological sample;
mixing the cell dispersion with a stock solution of paraformaldehyde in an amount that results in a final paraformaldehyde concentration of about 0.2 to 0.5%, forming a dispersion of fixed cells that maintain cellular and nucleic acid integrity; and
amplifying one or more target nucleic acid from one or more of the fixed cells.

2. The method of claim 1, wherein the final paraformaldehyde concentration is about 0.2%.

3. The method of claim 1, wherein the cells are compatible with immunostaining or immunoaffinity isolation and analysis.

4. The method of claim 1, wherein the biological sample is a tissue dispersion or a biological fluid.

5. The method of claim 3, wherein the biological fluid is a urine sample.

6. The method of claim 5, wherein the urine sample is a post digital rectal examination (DRE) urine sample.

7. The method of claim 1, wherein the stock solution of paraformaldehyde has a concentration of about 3 to 6% paraformaldehyde.

8. The method of claim 1, wherein the stock solution of paraformaldehyde has a concentration of about 4% paraformaldehyde.

9. The method of claim 1, further comprising performing nucleic acid analysis within 36 hours of sample processing.

10. The method of claim 1, wherein the cell dispersion is isolated using microfiltration, micromanipulation, microinjection, or combinations thereof.

11. A method for fixing cells comprising suspending cells in a stock solution of paraformaldehyde, wherein the solution once the cells are suspended comprises 0.2 to 0.5% paraformaldehyde.

12. The method of claim 11, wherein the cells are from a tissue dispersion or a biological fluid.

13. The method of claim 12, wherein the biological fluid is urine.

14. The method of claim 11, wherein the stock solution of paraformaldehyde has a pH of about 7.4.

15. The method of claim 11, wherein the stock solution of paraformaldehyde further comprises a pH stabilizing reagent.

16. The method of claim 11, wherein the stock paraformaldehyde solution is mixed with a sample within about 10 minutes to 3 hour of collecting a sample from a subject.

17. The method of claim 11, wherein the stock paraformaldehyde solution is mixed with a sample about 20 minutes after collecting the sample from a subject.
